(19) United States
(12) Patent Application Publication
(10) Pub. No.: US 2010/0227376 A1
(43) Pub. Date: Sep. 9, 2010

(54) METHOD OF OBTAINING BACTERIOPHAGE PREPARATION

(76) Inventors: Janusz Boratynski, Wroclaw (PL); Andrzej Gorski, Wroclaw (PL); Beata Weber-Dabrowska, Wroclaw (PL); Bozena Szermer-Olearnik, Wroclaw (PL)

Correspondence Address: COOPER & DUNHAM, LLP, 30 Rockefeller Plaza, 20th Floor, NEW YORK, NY, 10112, US

(21) Appl. No.: 12/452,420

(22) PCT Filed: Jun. 26, 2008
(86) PCT No.: PCT/PL2008/000045

§ 371 Date: Apr. 19, 2010

(30) Foreign Application Priority Data
Jun. 29, 2007 (PL) P 382800

(51) Int. Cl. C12N 7/02

(52) U.S. Cl. 435/239

(57) ABSTRACT

New method of removing pyrogens from bacteriophage preparations based on the natural processes of endotoxin degradation, and consisting of several stages, including in particular the addition of metal ions, the heterophase extraction, and chromatography.

### FIELD OF THE INVENTION

The present invention relates to a method of obtaining bacteriophage preparations containing trace content of endotoxins. Particularly, the present invention is related to a method of removing endotoxins from basteriophage lysate.

### BACKGROUND OF THE INVENTION

The commonly used antibiotic treatments have caused the micro-organisms to become less sensitive to antibiotics, including vancomycin—an antibiotic that disturbs the biosynthesis of peptidoglycan. Presently, the amount of information referring to alternatives to antibiotics, like the use of bacteriophages, grows rapidly. One of those who opt for such alternatives is Prof. Lederberg, a Nobel Prize Winner. His recent statement refers to the use of bacteriophages in bacterial infections therapy. The therapeutic use of bacteriophages is being investigated in several centres in the world and there will be more and more research into this topic. Institutions with the longest phage therapy tradition are the George Eliava Institute of Bacteriophage, Microbiology and Virology in Tbilisi and the Wroclaw Institute of Immunology and Experimental Therapy of the Polish Academy of Sciences. Numerous new companies geared towards using phages in bacterial infections therapy are currently being established, for example: Novolytics, Do-CoopTechnologies Ltd, CEO Novolytics Ltd, Phico Therapeutics Ltd.

During the therapy bacteria are destroyed by bacteriophages multiplying in them in the lytic cycle. The multiplied daughter molecules of bacteriophages are released and then they continue to destroy the next generations of bacteria.

In the case of bacteriophage lysate production for technological purposes, it is not only the number of descendant phage particles but also the released bacteria components, such as nucleic acids, proteins and the cell wall components that are of significance. The major compounds of the cell wall of Gram-negative bacteria are lipopolysaccharides (of up to 70%, called also pyrogens or endotoxins), peptidoglycans and proteins.

The effective removal of pyrogens from bacterial lysates is a key requirement to obtain bacteriophage preparations dedicated to bacterial infections therapy. Inducing the production of interleukins, TNF, NO etc., endotoxins are strong immune system stimulants.

### STATE OF THE ART

The procedures for removal or isolation of endotoxins are based on organic solvent extraction, such as extraction with phenol solution in water (Westphal O., Lueritz O., Bister F. Über die Extraktion von Bakterien mit Phenol/Wasser, Z. Naturforsch. 7: 148-155, 1952), extraction with a mixture of acids and aliphatic amines (Patent application Publication U.S. 2007/0020292A1), or application of extraction and chromatographic methods (Patent Application publication U.S. 2007/0031447A1). The biological preparations were purified from endotoxin by the use the interaction of metal ions with proteins (patent U.S. Pat. No. 6,942,802 B2 Sep. 13, 2005; WO02083710A1), and by precipitation of endotoxins with alcohol and bivalent counterions (U.S. Pat. No. 5,039,610). The use of bivalent ions in combinations with alcohols, resins and detergents is a subject of many patents, e.g. EPO 407037B1. Isolated crab lymph proteins were used to remove endotoxins (U.S. Pat. No. 5,760,177). Many methods of column chromatography were described. They use the lipopolysaccharide affinity for basic haptens, such as polymyxin [Petsch D, Beeskow T C, Anspach F B, Deckwer W D, (1997), Membrane absorbers for selective removal of bacterial endotoxin. J. Chromatogr B Biomed Sci Appl. 693(1):79-91)], resin based on calcium silicate [Hang J P, Wang Q, Smith T R, Hurst W E, Sulpizio T, (2005), Endotoxin removal using a synthetic adsorbent of crystalline calcium silicate hydrate. Biotechnol Prog. 21(4):1220-5)], synthetic polymers [Hirayama Ch, Sakata M, (2002), Chromatographic removal of endotoxin from protein solutions by polymer particles. Journal of Chromatography B, 781:419-432)], or polyanionic cellulose (Boratyński J, Syper D, Weber-Dabrowska B, Lusiak-Szelachowska M, Poźniak G, Górski A., Preparation of endotoxin-free bacteriophages, Cell Mol Biol Lett. 2004; 9(2):253-9).

### AIM OF THE INVENTION

The contemporary challenge is to win the race against micro-organisms that exchange genetic information among themselves, which allows them to become resistant to antibiotics. There is a constant need in the field to obtain bacteriophages preparations free from endotoxins and thus effective in bacterial infections therapy. Therefore, the aim of the invention is to obtain a sufficiently pure preparation of bacteriophages to be used for bacterial infections as an alternative of the antibiotics. In particular, the aim of the invention is to develop a method to remove endotoxins from bacteriophage lysates.

### DESCRIPTION OF THE INVENTION

It was observed that during the storage of bacteriophages the level of endotoxins determined with LAL (Limulus Amebocyte Lysate) test got reduced and then the reduction dynamics slowed down. It was assumed that endotoxins undergo destruction that may be caused by their natural instability. The destruction may be also supported by the enzymes in the bacteriophage lysates. Taking above into consideration, conditions were found, in which the destruction processes occur with high efficiency.

According to the invention, it was proved that, the concentration of toxins decreased to 5-10% as a result of natural destruction the speed of which is adjusted with metal ions, especially Mg²⁺. The next stage of the method is extraction from the broth containing bacteriophages of lipopolysaccharides and their fractions with the use of organic solvents that do not mix entirely with water. The solvents were alcohols and halogen derivatives.

Reduction of the content of substances giving positive results at the LAL test was observed in each examined sample after removing the traces of the extraction agent.

The present invention is related to a method of obtaining bacteriophage preparations containing trace content of endotoxins and is characterized in that the bacteriophage lysate obtained by use of a well known method is incubated in pH 4-10 environment, whereas the endotoxins contained in the bacteriophage lysate decompose during the incubation.

Preferably, the decomposition of endotoxins results from at least one of the following conditions: a natural process of biological decomposition of particles, and activity of the lysate enzymes that degrade endotoxins.

Preferably, the decomposition speed is favourably increased by incubation within the temperature range of 0 to 45° C., favourably at 14-37° C.

Even more preferably, the decomposition speed is favourably increased at magnesium ions presence, and at the same the content of calcium ions decreases.

In a preferred embodiment of the invention, the result of the self-destruction is reduction in the level of endotoxins denoted with the LAL (Limulus Amebocyte Lysate) test up to 95%, and the further self-destruction proceeds with low efficiency.

Preferably, the purified preparation is additionally mixed by shaking it with a not easily miscible organic solvent.

Preferably, the organic solvent is octyl alcohol or butyl alcohol.

Preferably, a water fraction of the bacteriophage lysate is separated after the lysate sedimentation.

Preferably, the separated water fraction is concentrated by evaporation of the excessive solvent or by ultrafiltration.

Preferably, trace impurities are eliminated from the concentrated bacteriophage preparation with organic solvent, favourably with octyl or butyl alcohol.

Preferably, the elimination of the trace amounts of organic solvents are eliminated by dialysis to ethanol water solutions with the concentration of 5-50%, and it is favourable to use ethanol solution with the concentration of 25%.

Preferably, the trace impurities are eliminated from the concentrated bacteriophage preparation by gel filtration of the bacteriophage preparations on molecular sieves, and the water solution of ethyl or isopropyl alcohol is used as an eluent, favourably 5% solution of isopropyl alcohol.

Preferably, the preparation purified with organic solvents is subjected to gel filtration, where the eluent is the solvent intended for storing of bacteriophage preparations.

Preferably, the high-molecular fraction containing purified bacteriophages is collected as the ultimate product.

### EXAMPLES

The bacteriophages tested were obtained by their cultivation on the target bacteria: Escherichia coli B for the T4 and HAP-1 bacteriophages, and Pseudomonas aeruginosa for the F-8 bacteriophage.

To do this, the relevant bacteria were multiplied at 37° C. in peptone water composed of: 0.4 g meat extract, 5.4 g enzymatic casein hydrolyzate, 1.7 g yeast hydrolyzate, 4.0 g peptone, 3.5 g NaCl dissolved in a 1 L of water. The previously prepared lysate containing appropriate bacteriophage was added to the suspension of target bacteria in their logarithmic growth phase. The level of endotoxin was determined by means of Limulus Amebocyte Lysate test (LAL).

### Example 1

The T4 bacteriophages were obtained by their cultivation on the Escherichia coli B bacteria at 37° C. 1.6 ml of the T4 bacteriophage lysate, previously dialysed against 0.04 M EDTA in 0.1 M sodium acetate pH 7.0 were added to the test tube containing 1.2 ml of 1-octanol. The sample was gently mixed on a rotator for 1 hour, then it was subjected to centrifugation (5 min, 3076 x g), and the collected lower aqueous phase was dialysed against 25% aqueous ethanol solution (the solution was changed 3 times). Before the biological testing, the sample was dialysed against 0.15 M NaCl (dialysis was changed 4 times). The level of endotoxins was determined with the LAL test was 15.7 EU/ml. The level of endotoxins in the initial lysate was 700 EU/ml. During the purification process of T4 bacteriophage loss of the lytic activity was below 10-fold.

### Example 2

The F-8 bacteriophages were obtained by their cultivation on the Pseudomonas aeruginosa bacteria at 37° C. 1.6 ml of the Ps-F8 bacteriophage lysate, previously dialysed against 0.04 M EDTA in 0.1 M sodium acetate pH 7.0 were added to the test tube containing 1.2 ml of 1-octanol. The sample was gently mixed on a rotator for 1 hour, next it was subjected to centrifugation (5 min, 3076 x g), and the collected lower aqueous phase was dialysed against 25% aqueous ethanol solution (the dialysis was changed 3 times). Before the biological testing, the sample was dialysed against 0.15 M NaCl (dialysis was changed 4 times). The level of endotoxins determined with the LAL test was 8.2 EU/ml. The level of endotoxins in the initial lysate was 380 EU/ml. During the F8 phage purification process there occurred a loss of the lytic activity below one order of magnitude.

### Example 3

The HAP-1 bacteriophages were obtained by their cultivation on the Escherichia coli B strain at 37° C. 1.6 ml of the HAP-1 bacteriophage lysate, previously dialysed against 0.04 M EDTA in 0.1 M sodium acetate pH 7.0 were added to the test tube containing 1.2 ml of 1-octanol. The sample was gently mixed on a rotator for 1 hour, next it was subjected to centrifugation (5 min, 3076 x g), and the collected lower aqueous phase was dialysed against 25% aqueous ethanol solution (the dialysis was changed 3 times). Before the biological testing, the sample was dialysed against 0.15 M NaCl (the dialysis was changed 4 times). The level of endotoxins was determined with the LAL test (14 EU/ml). The level of endotoxins in the initial lysate was 60,000 EU/ml. During the HAP-1 bacteriophage purification process there occurred a loss of the lytic activity below one order of magnitude.

### Example 4

The HAP-1 bacteriophages were obtained by their cultivation on the Escherichia coli B strain at 37° C. 1.6 ml of the HAP-1 bacteriophage lysate, previously dialysed against 0.03 M EDTA in 0.1 M sodium acetate pH 7.0 were added to the test tube containing 1.2 ml of 1-octanol. The sample was gently mixed on a rotator for 1 hour, next it subjected to centrifugation (5 min, 3076 x g), and the collected aqueous phase was dialysed against 25% aqueous ethanol solution (the dialysis was changed 3 times). Before the biological testing, the sample was dialysed against 0.15 M NaCl (the dialysis was changed 4 times). The level of endotoxins was determined with the LAL test (6.3 EU/ml). The level of endotoxins in the initial lysate was 1950 EU/ml. During the HAP-1 phage purification process 10-fold loss of the lytic activity was observed.

### Example 5

The T4 bacteriophages were obtained by their cultivation on the Escherichia coli B strain at 37° C. 1.6 ml of the T4 bacteriophage lysate containing 0.02 M MgCl₂ were added to the test tube containing 1.2 ml of 1-octanol. The sample was gently mixed on a rotator for 1 hour, next it was subjected to centrifugation (5 min, 3076 x g), and the collected aqueous phase was dialysed against 25% aqueous ethanol solution (the dialysis was changed 3 times). Before the biological testing, the sample was dialysed against 0.15 M NaCl (the dialysis was changed 4 times). The level of endotoxins was determined with the LAL test (5.4 EU/ml). The level of endotoxins in the initial lysate was 8700 EU/ml. During the T4 phage purification process 10-fold loss of the lytic activity was observed.

### Example 6

The F-8 bacteriophages were obtained by their cultivation on the Pseudomonas aeruginosa strain at 37° C. 1.6 ml of the F-8 bacteriophage lysate with 0.02 M MgCl₂ were added to the test tube containing 1.2 ml of 1-octanol. The sample was gently mixed on a rotator for 1 hour, next it was subjected to centrifugation (5 min, 3076 xg), and the collected lower aqueous phase was dialysed against 25% aqueous ethanol solution (the dialysis was changed 3 times). Before the biological testing, the sample was dialysed against 0.15 M NaCl (the dialysis was changed 4×). The level of endotoxins was determined with the LAL test (9 EU/ml). The level of endotoxins in the initial lysate was 1200 EU/ml. During the T4 phage purification process 10-fold loss of the lytic activity was observed.

### Example 7

The T4 bacteriophages were obtained by their cultivation on the Escherichia coli B strain at 37° C. 1.6 ml of the T4 bacteriophage lysate with 0.04 M MgCl₂ were added to the test tube containing 1.2 ml of 1-butanol. The sample was gently mixed on a rotator for 1 hour, next it was subjected to centrifugation (5 min, 3076 xg), and the collected aqueous phase was dialysed to 25% of ethanol water solution (the dialysis was changed 3 times). Before the biological testing, the sample was dialysed against 0.15 M NaCl (the dialysis was changed 4 times). The level of endotoxins was determined with the LAL test (160 EU/ml). The level of endotoxins in the initial lysate was 22 200 EU/ml. During the T4 phage purification process 5-fold loss in the lytic activity was observed.

### Example 8

The T4 bacteriophages were obtained by their cultivation on the Escherichia coli B strain at 37° C. 1.6 ml of the T4 bacteriophage lysate, previously dialysed against 0.04 M EDTA in 0.1 M sodium acetate pH 7.0 were added to the test tube containing 1.2 ml of 1-butanol. The sample was gently mixed on a rotator for 1 hour, next it was subjected to centrifugation (5 min, 3076 xg), and the collected lower aqueous phase was dialysed against 25% aqueous ethanol solution (the dialysis was changed 3 times). Before the biological testing, the sample was dialysed against 0.15 M NaCl (the dialysis was changed 4 times). The level of endotoxins was determined with the LAL test (140 EU/ml). The level of endotoxins in the initial lysate was 1950 EU/ml. During the T4 phage purification process 5-fold loss in the lytic activity was observed.

### Example 9

The T4 bacteriophages were obtained by their cultivation on the Escherichia coli B strain at 37° C. 1.6 ml of the T4 bacteriophage lysate, previously dialysed against 0.04 M EDTA in 0.1 M sodium acetate pH 7.0 were added to the test tube containing 1.2 ml of 1-octanol. The sample was gently mixed on a rotator for 1 hour, next it was subjected to centrifugation (5 min, 3076 xg), and the collected aqueous phase was dialysed against 25% aqueous ethanol solution (the dialysis was changed 3 times). Before the biological testing, the sample was dialysed against 0.15 M NaCl (dialysis was changed 4 times). The level of endotoxins was determined with the LAL test (20 EU/ml). The level of endotoxins in the control sample was 24 000 EU/ml. During the T4 phage purification process there occurred a loss in the lytic activity 5-fold.

### Example 10

The T4 bacteriophages were obtained by their cultivation on the Escherichia coli B bacteria strain at 37° C. 1.6 ml of the T4 bacteriophage lysate in 0.04 M MgCl₂ were added to the test tube containing 1.2 ml of 1-octanol. The sample was gently mixed on a rotator for 1 hour, next it was subjected to centrifugation (5 min, 3076 xg), and the collected aqueous phase was dialysed against 25% aqueous ethanol solution (the dialysis was changed 3 times). Before the biological testing, the sample was dialysed against 0.15 M NaCl (dialysis was changed 4 times). The level of endotoxins was determined with the LAL test (47 EU/ml). The level of endotoxins in the initial lysate was 21 000 EU/ml. During the T4 phage purification process there occurred a loss of the lytic activity 5-fold.

### Example 11

The T4 Bacteriophage were obtained by their cultivation on the Escherichia coli B bacteria strain at 37° C., in 1 L of peptone water containing: 0.4 g meat extract, 5.4 g enzymatic casein hydrolyzate, 1.7 g yeast hydrolyzate, 4.0 g peptone, 3.5 g NaCl. 150 ml of 1-octanol were added to the flask containing 25 l of the T4 bacteriophage lysate containing 0.01 M EDTA and 0.01M MgCl₂, and the entire solution was extracted for 1.5 h. After the octanol layer had been separated, the aqueous fraction was dialysed against 10% isopropyl alcohol in combination with ultrafiltration. The next stage was gel filtration of the concentrated T4 bacteriophage in the presence of 5% isopropyl alcohol. The gel filtration was done on a column filled with S-200 Sephacryl, with capacity of 50 L. The level of endotoxins was determined with the LAL test and was 81 EU/ml. The level of endotoxins in the initial lysate was 5800 EU/ml. The phage titre in the purified preparation was 5.3×10¹².

### Example 12

The T4 bacteriophages were obtained by their cultivation on the Escherichia coli in peptone:water in the presence of 0.05 M MgCl₂ at 37° C. 50 L T4 bacteriophage lysate was gently mixed with 20 L of 1-butanol for 6 h at 33° C. In the next step butanol layer was separated and the aqueous fraction was dialysed against 0.15 M NaCl containing 15% ethanol in combination with ultrafiltration (25× concentration). The next stage was gel filtration of concentrated bacteriophages in the presence of 15% ethyl alcohol and 0.15 NaCl on S-200 Sephacryl column (capacity 50 L). The endotoxin level in the initial lysate was 36700 EU/ml. In the purified T4 bacteriophage fraction the level of endotoxin was 14 EU/ml. During the T4 purification process titer of bacteriophage was decreased 5-fold.

1. A method of obtaining bacteriophage preparations containing trace content of endotoxins comprising incubating a bacteriophage lysate in pH 4-10 environment, whereas the endotoxins contained in the bacteriophage lysate decompose during the incubation.

2. Method according to claim 1, wherein the decomposition of endotoxins results from at least one of the following conditions: a natural process of biological decomposition of particles, and activity of the lysate enzymes that degrade endotoxins.

3. Method according to claim 1, wherein the decomposition speed is favourably increased by incubation within the temperature range of 0 to 45° C.

4. Method according to claim 1, wherein the decomposition speed is favourably increased at magnesium ions presence, and at the same the content of calcium ions decreases.

5. Method according to claim 1, wherein the result of the self-destruction is reduction in the level of endotoxins denoted with the LAL (Limulus Amebocyte Lysate) test up to 95%, and the further self-destruction proceeds with low efficiency.

6. Method according to claim 1, wherein the purified preparation is additionally mixed by shaking it with a not easily miscible organic solvent.

7. Method according to claim 6, wherein the organic solvent is octyl alcohol or butyl alcohol.

8. Method according to claim 1, wherein a water fraction of the bacteriophage lysate is separated after the lysate sedimentation.

9. Method according to claim 8, wherein the separated water fraction is concentrated by evaporation of the excessive solvent or by ultrafiltration.

10. Method according to claim 9, wherein trace impurities are eliminated from the concentrated bacteriophage preparation with organic solvent.

11. Method according to claim 10, wherein the elimination of the trace amounts of organic solvents are eliminated by dialysis to ethanol water solutions with the concentration of 5-50%.

12. Method according to claim 9, wherein the trace impurities are eliminated from the concentrated bacteriophage preparation by gel filtration of the bacteriophage preparations on molecular sieves, and the water solution of ethyl or isopropyl alcohol is used as an eluent, favourably 5% solution of isopropyl alcohol.

13. Method according to claim 10, wherein the preparation purified with organic solvents is subjected to gel filtration, where the eluent is the solvent intended for storing of bacteriophage preparations.

14. Method according to claim 1, further comprising collecting a high-molecular fraction containing purified bacteriophages as the ultimate product.

15. The method of claim 3, wherein the incubation is at a temperature of 14-37° C.

16. The method of claim 10, wherein the organic solvent is octyl or butyl alcohol.
